(19) United States
(12) Patent Application Publication
(10) Pub. No.: US 2006/0052760 A1
(43) Pub. Date: Mar. 9, 2006

(54) Bone dust accumulator

(76) Inventor: Ulrich Batzdorf, Los Angeles, CA (US)

Correspondence Address: NIXON & VANDERHYE, PC, 901 NORTH GLEBE ROAD, 11TH FLOOR, ARLINGTON, VA, 22203, US

(21) Appl. No.: 11/170,135

(22) Filed: Jun. 30, 2005

Related U.S. Application Data
(60) Provisional application No. 60/603,010, filed on Aug. 20, 2004.

(51) Int. Cl. A61M 1/00

(52) U.S. Cl. 604/319

(57) ABSTRACT

A method and apparatus for accumulating bone dust in a fluid from a surgical site or the like, wherein a collecting vessel is provided that has an inlet opening near one end thereof and an outlet opening near the opposite end thereof. A permeable plunger is slidably mounted in the vessel for movement between a first position wherein it is disposed between the inlet opening and the outlet opening, and a second position wherein it is disposed near the one end of the vessel. Fluid containing bone dust from a surgical site is conveyed through the inlet opening into the vessel when the plunger is in the first position, such that the fluid passes through the plunger and through the outlet opening. The plunger is of a construction to prevent the bone dust from passing therethrough such that it is accumulated between the plunger and the one end of the vessel. When sufficient bone dust is accumulated, the plunger is moved to the second position to move the accumulated bone dust toward the one end of the vessel for removal therefrom.

This application claims the priority of U.S. Provisional Application No. 60/603,010, filed on Aug. 20, 2004.

### BACKGROUND OF THE INVENTION

1. Field of the Invention

The present invention relates to a method and apparatus for collecting bone dust which is generated by the drilling of bone during an operation or the like and, more particularly, to such a method and apparatus wherein the accumulated bone dust can be used as part of the bone materials for bone fusion or other purposes.

2. Description of Related Art

Many surgical procedures are performed which involve drilling away bone as, for example, for decompression of the spinal cord or nerve roots, as well as in the preparation of other bones in the body for fusion or other purposes. High-speed drills, burrs or other tools are generally employed for this purpose, and the bone dust so generated is usually suctioned off together with irrigating fluid and discarded. Once the preparation of the bone has been completed, the fusion is performed. This often involves placement of a metallic stabilizer with screws or other anchoring devices, and the addition of bone chips or bone struts to bond, in time, with the adjacent host bone to form a solid mass. Fragments of bone which have been removed early in the procedure are used, often with bone from a bone bank, to provide the bone stock for the fusion. Various additives, including bone morphogenetic protein (BMP) are now available to enhance the bone fusion process.

It is believed that bone dust would augment the fusion process because of the consistency and particle size, allowing it to flow between the larger bone chips, and because of its content of BMP. Bone dust could be used alone or with a bonding agent, such as fibrin glue, which is commercially available. Advantages of using the patient's own bone dust include cost and safety with respect to transmissible agents and foreign antigens.

The following patents and patent application publication disclose different types of apparatus for collecting bone particles or fragments from a surgical bone site during a surgical procedure:

U.S. Pat. No. 5,954,961 discloses a bone particle collection apparatus and method wherein bone particle containing effluent is received into a screen basket assembly, and suction is applied to the basket assembly to remove liquid which passes therethrough. The screen basket assembly is then removed and a plunger is inserted into an open end thereof to consolidate the bone particles and expel them onto a collection surface or the like.

U.S. Pat. No. 6,299,763 discloses a bone and cell filter trap wherein air flow carrying liquids, solids and gases from a patient's surgical site is forced to pass through a cylindrically-shaped mesh which is sized such that only autogenous bone and cell marrow and blood elements are trapped in the mesh. Upon completion of the medical procedure, the mesh is removed from the inlet and outlet housings and a plunger device is pushed up and down inside the mesh open end to dislodge the elements trapped therein.

U.S. Pat. No. 6,468,225 discloses a method and apparatus for collecting bone tissue fragments wherein the fragments are collected from liquid fluid being evacuated by suction from a surgical operation in bone tissue. The liquid is allowed to pass into a cylindrical sieve through one open end thereof and through the sieve wall from the inside to the outside thereof, while bone tissue fragments are collected on the inside surface of the sieve and are scraped off and deposited outside the sieve.

Patent application Publication No. 2003-0130594 A1 discloses an autogenous bone collection and delivery system wherein suction is applied through a filter cartridge and autogenous bone is collected on a filter medium within the cartridge. The cartridge is then removed and placed into a delivery device having a rachet system that actuates a plunger base to accurately extrude the bone material from the filter cartridge into the desired surgical location.

The above-described patents and patent application publication all disclose apparatus that would not be suitable for the collection or accumulation of bone dust and is so constructed that a collection screen, basket, mesh or sieve on which the bone fragments are accumulated must be removed from the apparatus so that the bone fragments can be removed therefrom by a plunger, scraper of the like. Such apparatus is complicated in construction, difficult and/or expensive to manufacture and difficult to use.

The new and improved method and apparatus of the present invention can be used to collect bone dust in an effective and simple manner, and is not subject to the disadvantages of the above-described prior art apparatus.

### SUMMARY OF THE INVENTION

The bone dust accumulator apparatus of the present invention comprises a sterile collecting vessel having an inlet opening and tube near one end thereof and an outlet opening and tube near the opposite end thereof. The tubes are suitable for connection to suction tubing or the like. The collecting vessel has end caps or the like mounted on the end portions thereof to seal the vessel. The vessel is separated into two chambers by a permeable plunger that is disposed between the inlet and outlet openings and is slidably movable within the vessel by means of an actuating rod extending out of one end of the vessel. The plunger has openings therethrough that are of a size to prevent bone dust of a predetermined size or a greater size from passing therethrough.

In operation, the bone dust accumulator apparatus is attached at a convenient location in the irrigating fluid suction line, with the input tubing coming from the operating site being attached to the inlet tube, and outlet tubing being attached to the outlet tube. During the bone dust collection process, fluid containing bone dust enters the inlet opening and the vessel in a first chamber between the permeable plunger and the one end portion of the vessel. Bone dust accumulates in the first chamber and the fluid passes through the plunger, enters a second chamber between the plunger and the opposite end of the vessel, and passes through the outlet opening and tube.

When sufficient bone dust has been collected, the incoming flow of bone dust and fluid is terminated, and the plunger is moved toward the one end of the vessel to concentrate and somewhat compress the harvested bone dust. The adjacent end cap is then opened or removed and the plunger is further depressed to expel the bone dust into a suitable collection device such as a separate collecting cup. The same or a new end cap can then be re-attached to the adjacent vessel end portion to continue harvesting bone dust.

### BRIEF DESCRIPTION OF THE DRAWINGS

FIG. 1 is a side elevational view of one embodiment of the bone dust accumulator apparatus of the present invention, with the plunger thereof in a first position to enable the accumulation of bone dust; and

FIG. 2 is a side elevational view similar to FIG. 1 with the plunger in a second position wherein the accumulated bone dust is moved and compressed for collection at one end of the apparatus.

### DETAILED DESCRIPTION OF THE INVENTION

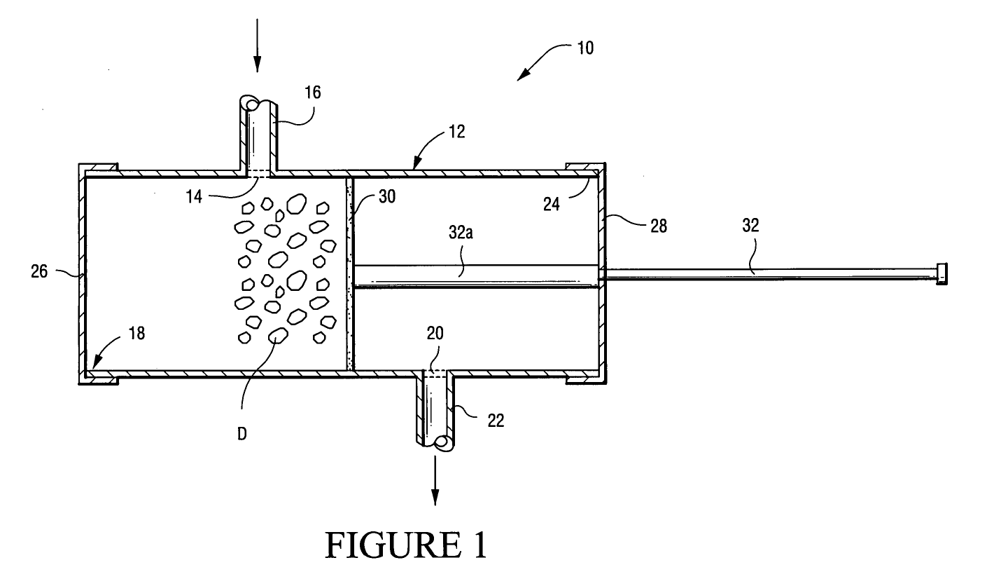

Referring to FIG. 1, the bone dust accumulator apparatus 10 of the present inventions comprises a collecting vessel 12 of any suitable construction and formed of any suitable material, such as metal or plastic. As an illustrative example, the vessel 12 may be of elongated, cylindrical construction. The vessel 12 may be treated in any appropriate manner so as to be sterile.

The collecting vessel 12 comprises an inlet opening 14 and inlet tube 16 near one end 18 thereof, and an outlet opening 20 and outlet tube 22 near the opposite end 24 thereof. The one end 18 of the vessel 12 is closed by an end closure 26 of any suitable construction which may be movably mounted on one end 18 to open and close it, or may be removably mounted thereon for the opening and closing thereof. The opposite end 24 of the vessel 12 is closed by an end closure 28 of any suitable construction.

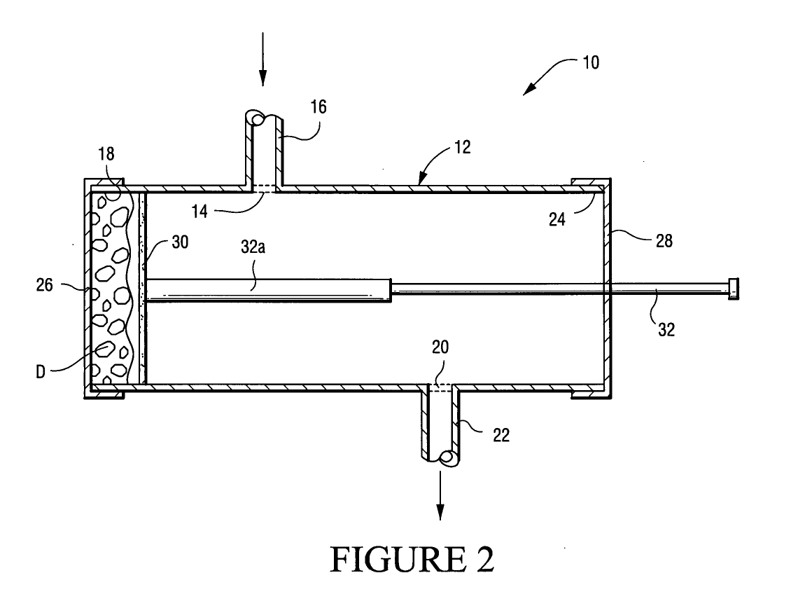

A plunger 30 formed of a suitable permeable material, e.g., a biologically inert plastic, such as used in syringes, or a biologically inert metal such as stainless steel, is slidably mounted in the collecting vessel 12 and is movable between a first position shown in FIG. 1, wherein it is disposed between the inlet opening 14 and the outlet opening 20, and a second position shown in FIG. 2 wherein it is disposed near the one end 18 of the vessel 12. An actuating rod 32 is connected to the plunger 30 and extends outwardly of and is in slidable engagement with the end closure 28 at the opposite end 24 of the vessel 12. The inner portion 32a of the actuating rod 32 is of a larger width or diameter so as to provide a stop that engages the end closure 28 to limit the movement of the plunger 30 in a direction toward the end closure 28.

The inlet tube 16 is constructed to be connected to a suction tube (not shown) or the like for conveying a fluid containing bone dust from a surgical site or the like in the direction of the arrow shown in FIG. 1. The openings through the permeable plunger 30 are of a size to permit the incoming fluid to pass therethrough and to exit the outlet tube 22, and to prevent the passage of the incoming bone dust therethrough such that bone dust D accumulates in the portion of the vessel 12 between the plunger 30 and the end closure 26 at the one end 18 of the vessel 12. As an illustrative example, the size of the openings through the permeable plunger 30 may be in the range of 1 mm to 2.5 mm. The outlet tube 22 is constructed to be connected to suitable suction tubing (not shown) or the like for the purpose of removing the fluid from the vessel 12.

When sufficient bone dust has accumulated in the portion of the vessel 12 between the plunger 30 and the end closure 26, the flow of incoming fluid and bone dust is terminated and the plunger 30 is moved to the second position shown in FIG. 2 to move the bone dust D toward the one end 18 of the vessel 12 and to concentrate or compress it near the end closure 26. The end closure 26 may then be opened or removed from the one end 18 of the vessel 12 to enable the accumulated bone dust to be removed from the vessel 12 by further movement of the plunger 30 toward the open end 18. After the bone dust is removed, the end closure 26 is closed or again mounted on the one end 18 of the vessel 12, and the plunger 30 is moved back to the first position shown in FIG. 1 to begin a new cycle of accumulating bone dust in the vessel 12.

If necessary, a stand or other support (not shown) may be provided to maintain the collecting vessel 12 in an upright or operable position as shown in FIGS. 1 and 2.

As will be readily seen from the foregoing description, the bone dust accumulating apparatus 10 of the present invention is simple in construction, inexpensive to manufacture and reliable in operation to enable bone dust to be accumulated therein and easily removed therefrom.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. Apparatus for accumulating bone dust in a fluid from a surgical site or the like, comprising:
a collecting vessel having an inlet opening near one end thereof and an outlet opening near the opposite end thereof;
a first end closure being mounted on said one end of said vessel;
said opposite end of said vessel being closed;
a plunger being slidably mounted in said vessel for movement between a first position wherein it is disposed between said inlet opening and said outlet opening, and a second position wherein it is disposed near said one end of said vessel;
said plunger being permeable and being constructed to allow the fluid to pass therethrough and prevent the bone dust from passing therethrough;
whereby when said plunger is in said first position bone dust in a fluid entering said inlet opening accumulates in said vessel between said plunger and said one end of said vessel, the fluid passes through said plunger and said outlet opening, and the accumulated bone dust is moved toward said one end when said plunger is moved toward said second position for removal of the bone dust from said vessel when said first end closure is opened or removed therefrom.

2. The apparatus of claim 1, wherein said collecting vessel is of elongated, generally cylindrical construction.

3. The apparatus of claim 2, wherein said collecting vessel is formed of metal or plastic.

4. The apparatus of claim 1, wherein said inlet opening is in an inlet tube extending outwardly from said collecting vessel, and said outlet opening is in an outlet tube extending outwardly from said collecting vessel.

5. The apparatus of claim 4, wherein said inlet tube and said outlet tube are constructed to be connected to suction tubing or the like.

6. The apparatus of claim 1, wherein said plunger is disposed between said one end and said inlet opening when in said second position.

7. The apparatus of claim 1, wherein an actuating rod is connected to said plunger and is in slidable engagement with and extends outwardly of said opposite end of said vessel.

8. The apparatus of claim 1, wherein said plunger has openings therethrough in a size range from 1 mm to 2.5 mm.

9. The apparatus of claim 1, wherein said plunger is formed of a biologically inert plastic or metal.

10. The apparatus of claim 1 wherein said end closure is movably mounted on said one end of said vessel.

11. The apparatus of claim 10, wherein said end closure is removably mounted on said one end of said vessel.

12. The apparatus of claim 7, wherein said actuating rod has a stop to limit the movement of said plunger toward said opposite end of said vessel.

13. A method of accumulating bone dust in a fluid from a surgical site or the like, comprising:
providing a collecting vessel having an inlet opening near one end thereof and an outlet opening near the opposite end thereof, and a permeable plunger slidably mounted in said vessel for movement between a first position wherein it is disposed between said inlet opening and said outlet opening, and a second position wherein it is disposed near said one end of said vessel;
conveying fluid containing bone dust through said inlet opening into said vessel when said plunger is in said first position such that fluid passes through said plunger and through said outlet opening, said plunger being of a construction to prevent the bone dust from passing therethrough; and
moving said plunger to said second position wherein the accumulated bone dust is moved toward said one end of said vessel for removal therefrom.

14. The method of claim 13 wherein an end closure is provided on said one end of said vessel and is movable to an open position to facilitate the removal of bone dust therefrom.

15. The method of claim 13 wherein said plunger has openings therethrough in a size range of 1 mm to 2.5 mm.

16. The method of claim 13 wherein said plunger is moved by an actuating rod connected thereto and extending outwardly of said opposite end of said vessel.
